(19) United States
(12) Patent Application Publication
(10) Pub. No.: US 2010/0247617 A1
(43) Pub. Date: Sep. 30, 2010

(54) Transdermal Administration Of (2S)-(4E)-N-Methyl-5-(3-(5-Isopropoxypyridin)yl)-4-Penten-2-Amine

(75) Inventors: Merouane Bencherif, Winston-Salem, NC (US); John W. James, Winston-Salem, NC (US); Michael G. Williams, Winston-Salem, NC (US); Johannes Antonius Maria Neelissen, Sodertalje (SE)

Correspondence Address: Pepper Hamilton LLP, 400 Berwyn Park, 899 Cassatt Road, Berwyn, PA, 19312-1183, US

(73) Assignees: TARGACEPT, INC., Winston-Salem (US); ASTRAZENECA AB, Sodertalje (SE)

(21) Appl. No.: 12/671,232

(22) PCT Filed: Jul. 30, 2008
(86) PCT No.: PCT/US2008/071638

§ 371 Date: May 17, 2010

Related U.S. Application Data
(60) Provisional application No. 60/953,062, filed on Jul. 31, 2007.

(51) Int. Cl. A61K 31/44; A61K 9/70; A61P 25/00; A61P 29/00

(52) U.S. Cl. 424/449; 514/349

(57) ABSTRACT

The present invention generally relates to the transdermal administration of (2S)-(4E)-N-methyl-5-(3-(5-isopropoxypyridin)yl)-4-penten-2-amine, or pharmaceutically acceptable salts thereof. The transdermal administration can be effected using transdermal drug delivery devices, semi-solid dosage forms, or iontophoresis. The drug delivery devices and/or semi-solid dosage forms can provide instantaneous release, sustained release, or combinations thereof, and can include permeation enhancers and other components to assist in drug transport across the dermis, especially the epidermis. The compositions can be used to treat and/or prevent any indication which the active ingredients are capable of treating and preventing, but deliver (2S)-(4E)-N-methyl-5-(3-(5-isopropoxypyridin)yl)-4-penten-2-amine, or pharmaceutically acceptable salts thereof, in an efficacious manner. Disorders that can be treated and/or prevented include central nervous system disorders, addictions, pain, and inflammation.

### CROSS-RELATION TO RELATED APPLICATIONS

The present application claims benefit of U.S. Provisional Application 60/953,062, filed Jul. 31, 2007, herein incorporated by reference in its entirety.

### FIELD OF THE INVENTION

The present invention generally relates to the transdermal administration of (2S)-(4E)-N-methyl-5-(3-(5-isopropoxypyridin)yl)-4-penten-2-amine, or pharmaceutically acceptable salts thereof.

### BACKGROUND OF THE INVENTION

A variety of metanicotine analogs have been proposed for use in treating a variety of disorders, predominantly via oral administration. See, for example, U.S. Pat. No. 5,616,716, U.S. Pat. No. 5,861,423, U.S. Pat. No. 6,232,316 and U.S. Pat. No. 6,958,399, the contents of which are hereby incorporated by reference. However, some of these compounds suffer from relatively fast degradation in vivo, which makes it difficult to administer them to the site of action via routes that involve first pass metabolism in the liver. Even for metanicotine analogs that do not have rapid first pass metabolism, routes of administration other than the oral route can be advantageous, particularly if they provide improvements in therapeutic levels or the onset of activity.

It would be advantageous to provide new compositions and methods for administering certain metanicotine analogs. The present invention provides such compositions and methods.

### SUMMARY OF THE INVENTION

The present invention relates the transdermal delivery of 5-(3-(5-isopropoxypyridin)yl)-4-penten-2-amine, and pharmaceutically acceptable salts thereof (hereinafter referred to as active ingredient). (2S)-(4E)-N-methyl-5-(3-(5-isopropoxypyridin)yl)-4-penten-2-amine has acceptable bioavailability when administered orally, but transdermal administration provides other advantages over oral administration.

In some embodiments, the transdermal drug delivery is carried out using devices that include a polymeric barrier, adhered to the skin with a suitable adhesive, and which also include a suitable amount of the active ingredients, or salts thereof, in solution or dispersion and in contact with the skin or a rate-controlling membrane may be used between the active-containing composition and the skin. In others, the delivery is carried out using semisolid compositions, such as crèmes or lotions, which include the active ingredients, and which are applied to the skin. In still other embodiments, the active ingredients are delivered using iontophoresis, wherein the positively charged active agents are administered by electroosmosis. There may also be embodiments wherein the active ingredient(s) is formulated within the matrix of the adhesive.

The drug delivery devices and/or semi-solid dosage forms can provide instantaneous release, sustained release, or combinations thereof, and can include permeation enhancers and other components to assist in drug transport into the dermis (via transport across the stratum corneum of the epidermis) wherein the active ingredient is available for systemic absorption or local effects.

The transdermal delivery of (2S)-(4E)-N-methyl-5-(3-(5-isopropoxypyridin)yl)-4-penten-2-amine, or its pharmaceutically acceptable salts, can be used to treat and/or prevent any indication which the active ingredient is capable of treating and/or preventing, while delivering the active ingredient in an efficacious manner.

Some embodiments relate to methods involve treating or preventing central nervous system disorders. Central nervous system disorders include disorders characterized by dysfunction of nicotinic cholinergic neurotransmission, including disorders involving neuromodulation of neurotransmitter release, such as dopamine release. The central nervous system (CNS) disorders can be characterized by an alteration in normal neurotransmitter release. Other methods involve treating certain other conditions (e.g., alleviating pain and inflammation). The methods involve administering to a subject an effective amount of a composition as described herein via a transdermal route to treat or prevent the disorder, or to alleviate or eliminate the pain and/or inflammation.

The transdermal compositions can also be used in smoking cessation, ideally without the side effect profiles associated with nicotine patches. In addition to smoking cessation, the compositions can also be used to treat other addictions, such as addiction to alcohol, narcotics (including opiates such as oxycodone, morphine, heroine, and the like), prescription drugs, gambling, and the like.

The compositions for transdermal administration include an effective amount of (2S)-(4E)-N-methyl-5-(3-(5-isopropoxypyridin)yl)-4-penten-2-amine or its pharmaceutically acceptable salts, along with pharmaceutically acceptable carriers and/or excipients. The compositions can be in the form of patches containing the active ingredient, or a pharmaceutically acceptable salt thereof. The compositions can optionally include components such as permeation enhancers, adhesives, and means for providing instantaneous and/or sustained release of the active ingredients. The compositions may or may not include a rate-controlling membrane to modulate the release of the active ingredient from the device to the skin.

The pharmaceutical compositions include effective amounts of (2S)-(4E)-N-methyl-5-(3-(5-isopropoxypyridin)yl)-4-penten-2-amine, or its pharmaceutically acceptable salts. Accordingly, the compound is released from the composition, absorbed, and interacts with relevant nicotinic receptor sites of a subject to act as a therapeutic agent for treating or preventing a wide variety of conditions and disorders, particularly central nervous system disorders, (including those disorders characterized by an alteration in normal neurotransmitter release), pain, and/or inflammation.

The pharmaceutical compositions provide therapeutic benefit to individuals suffering from such disorders and exhibiting clinical manifestations of such disorders in that the active ingredients within those compositions, when employed in effective amounts, have the potential to (i) exhibit nicotinic pharmacology and affect relevant nicotinic receptor sites (e.g., act as a pharmacological agonist to activate nicotinic receptors), and (ii) elicit neurotransmitter secretion, and hence prevent and suppress the symptoms associated with those diseases. In addition, the active ingredients are expected to have the potential to (i) increase the number of nicotinic cholinergic receptors of the brain of the patient, (ii) exhibit neuroprotective effects and (iii) when employed in effective amounts do not cause appreciable adverse side effects (e.g., significant increases in blood pressure and heart rate, significant negative effects upon the gastro-intestinal tract, and significant effects upon skeletal muscle). The pharmaceutical compositions described herein are believed to be safe and effective with regards to treating and preventing a wide variety of conditions and disorders.

The scope of the present invention includes all combinations of aspects, embodiments, and preferences herein described. The foregoing and other aspects of the present invention are explained in detail in the detailed description and examples set forth below.

### BRIEF DESCRIPTION OF THE DRAWINGS

FIG. 1 illustrates the cumulative penetrated amounts and derived steady state fluxes for tested concentrations of compositions of the present invention during a 24-hour period.

### DETAILED DESCRIPTION OF THE INVENTION

The present invention relates to the transdermal administration of (2S)-(4E)-N-methyl-5-(3-(5-isopropoxypyridin)yl)-4-penten-2-amine, and pharmaceutically acceptable salts thereof. The term “transdermal delivery” as used herein means a method for drug absorption through the skin, or across the stratum corneum of the epidermis and into the dermis for absorption into the systemic circulation or for local effects.

The transdermal route can be effective in delivering (2S)-(4E)-N-methyl-5-(3-(5-isopropoxypyridin)yl)-4-penten-2-amine, which exhibits high affinity and selectivity for, and activity at the α4β2 neuronal nicotinic receptor (NNR) subtype.

The transdermal route can also be more effective than the oral route in that it can provide for relatively faster or slower (extended) absorption and onset of therapeutic action.

Further, the route may be preferred and lead to improved compliance for patients who have difficulty in swallowing tablets, capsules, or other solids (for example, the elderly, the young, and those with impairments, such as dementia), those who have intestinal failure, those whose schedules do not permit dosing throughout the day, and those in extended care facilities.

Accordingly, there are many advantages to the transdermal administration of (2S)-(4E)-N-methyl-5-(3-(5-isopropoxypyridin)yl)-4-penten-2-amine, and/or its pharmaceutically acceptable salts (herein collectively referred to as “active ingredients” or “active agents”).

The present invention will be better understood with reference to the following definitions:

The terms “active ingredient”, “drug” or “active drug” or “active agent” mean (2S)-(4E)-N-methyl-5-(3-(5-isopropoxypyridin)yl)-4-penten-2-amine, as well as any prodrugs thereof and pharmaceutically acceptable salts, hydrates, cocrystals, and solvates of the active ingredient and the prodrugs.

The term “other ingredients” means any excipients, diluents, binders, lubricants, carriers, surfactants, and mixtures thereof that are formulated with the active ingredient.

The term “appropriate period of time” or “suitable period of time” means the period of time necessary to achieve a desired effect or result. For example, a mixture can be blended until a potency distribution is reached that is within an acceptable range for a given application or use of the blended mixture.

“Carriers” or “vehicles” as used herein refer to carrier materials suitable for transdermal drug administration, and include any such materials known in the art, e.g., any liquid, gel, solvent, liquid diluent, solubilizer, or the like, which is non toxic and which does not interact with other components of the composition in a deleterious manner. Examples of suitable vehicles for use herein include water, alcohols such as isopropyl alcohol and isobutyl alcohol, polyalcohols such as glycerol, and glycols such as propylene glycol, and esters of such polyols, (e.g., mono-, di-, or tri-glycerides).

By “controlled” is meant a metered or measured release of the active ingredient to reduce or minimize peak and valley exposure cycles in blood, plasma, or other biological fluids normally present in some routes of administration of a pharmacologically active agent.

An “effective” or an “adequate” permeation enhancer as used herein means a permeation enhancer that will provide the desired increase in skin permeability and correspondingly, the desired depth of penetration, rate of administration, and amount of active ingredient delivered.

The term “effective amount,” as used herein means the amount determined by such considerations as are known in the art for treating or preventing central nervous system disorders, or treating addiction, inflammation or pain in an individual, wherein it must be effective to provide measurable relief in treated individuals such as exhibiting improvements including, but not limited to, more rapid recovery, a more sustained recovery, improvement or elimination of symptoms or reduction of complications, or other measurements as appropriate and known to those skilled in the medical arts.

“Penetration enhancement” or “permeation enhancement” as used herein relates to an increase in the permeability of skin to a pharmacologically active agent, namely, so as to increase the rate at which the active ingredient permeates through the skin (i.e., flux) and enters the bloodstream or the local site of action. The enhanced permeation effected by using these enhancers can be observed by measuring the rate of diffusion (or flux) of active ingredient through animal or human skin or a suitable polymeric membrane using a diffusion cell apparatus as described in the examples herein.

By “transdermal” delivery, applicants intend to include both transdermal and percutaneous administration, i.e., delivery by passage of an active ingredient through the skin and into the bloodstream.

By “sustained” is meant extended maintenance of steady state plasma levels of an active ingredient.

The term “unit dose,” “unit dosage,” or “unit dosage form” means a physically discrete unit that contains a predetermined quantity of active ingredient calculated to produce a desired therapeutic effect. The dosage form can be in any suitable form for transdermal administration, which forms are well known to those of skill in the art.

In any embodiments described herein, the active blend of a dosage form generally includes one or more pharmaceutically acceptable adhesives, excipients, carriers, diluents, binders, lubricants, glidants, or disintegrants and depends upon the purpose for which the active ingredient is being applied. In general, transdermal compositions include other ingredients including, but not limited to, excipients, diluents, carriers, permeation enhancers, and mixtures thereof.

The active ingredient, (2S)-(4E)-N-methyl-5-(3-(5-isopropoxypyridin)yl)-4-penten-2-amine, its pharmaceutically acceptable salts, methods for their synthesis, transdermal compositions including these active ingredients, and methods of treatment using the ingredients and compositions, are described in more detail below.

### I. Metanicotine Compounds

The active ingredients that are the subject of the present invention are (2S)-(4E)-N-methyl-5-(3-(5-isopropoxypyridin)yl)-4-penten-2-amine, and pharmaceutically acceptable salts thereof. The formula for the free base is shown below:

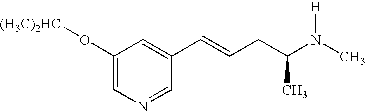

U.S. Pat. No. 6,958,399 describes (2S)-(4E)-N-methyl-5-(3-(5-isopropoxypyridin)yl)-4-penten-2-amine, and pharmaceutically acceptable salts thereof and is herein incorporated by reference.

### Salts of the Active Ingredients

The ideal pH for transdermal drug delivery is typically in the range of from about 3.5 to about 9.5. Since the instant active ingredient is itself basic (i.e., it includes two basic nitrogen atoms), it tends to produce solutions in the upper end of this pH range when the free base form is placed in water. For this reason, it may be desirable to neutralize at least one of the nitrogens, using a pharmaceutically acceptable acid to form one or more pharmaceutically acceptable salt of the free base.

Examples of suitable pharmaceutically acceptable salts include inorganic acid addition salts such as chloride, bromide, sulfate, phosphate, and nitrate; organic acid addition salts such as acetate, galactarate, propionate, succinate, lactate, glycolate, malate, tartrate, citrate, maleate, fumarate, methanesulfonate, p-toluenesulfonate, benzoate, hydroxybenzoate, edisylate, orotate, R-mandelate and ascorbate; salts with amino acids such as lysine monohydrochloride, aspartate and glutamate. The salts may be in some cases hydrates or ethanol solvates. Representative salts are provided as described in U.S. Pat. No. 5,597,919 to Dull et al., U.S. Pat. No. 5,616,716 to Dull et al. and U.S. Pat. No. 5,663,356 to Ruecroft et al., each herein incorporated by reference.

In some embodiments, the acids also help with the permeability of the active agent through the skin. That is, when the neutralizing agent is a fatty acid, it can neutralize one or both of the basic nitrogen atoms on the active ingredient, while at the same time increasing the permeability of the active ingredient through the skin. Thus, in one embodiment, the active ingredient is present as a fatty acid salt, wherein one or both of the basic nitrogens is reacted with the acid group in the fatty acids to form a salt. Accordingly, the salts can be present in a 1:1 or 1:2 molar ratio of active ingredient to fatty acid. The reaction of a fatty acid and an amine to form a fatty acid carboxylate salt is well known to those of skill in the art. The fatty acids typically have between 4 and 20 carbon atoms, and, more typically between 10 and 18 carbon atoms. Stearic acid is one representative fatty acid.

### II. Active Ingredient Preparation

The synthesis of (2S)-(4E)-N-methyl-5-(3-(5-isopropoxypyridin)yl)-4-penten-2-amine can be carried out, for example, using procedures described in U.S. Pat. No. 6,958,399, as noted above herein incorporated by reference. The synthesis of salts of (2S)-(4E)-N-methyl-5-(3-(5-isopropoxypyridin)yl)-4-penten-2-amine can be accomplished by combining (2S)-(4E)-N-methyl-5-(3-(5-isopropoxypyridin)yl)-4-penten-2-amine with various inorganic and organic acids in appropriate solvents, as exemplified in U.S. Pat. No. 6,432,954 and PCT WO06/053082, each incorporated by reference. The hemigalactarate salt of (2S)-(4E)-N-methyl-5-(3-(5-isopropoxypyridin)yl)-4-penten-2-amine can be prepared using the techniques set forth in U.S. Pat. No. 6,958,399, as noted above herein incorporated by reference.

### III. Transdermal Compositions

The compositions for transdermal administration include (2S)-(4E)-N-methyl-5-(3-(5-isopropoxypyridin)yl)-4-penten-2-amine, or its pharmaceutically acceptable salts, including fatty acid salts, and optionally can also include other ingredients including, but not limited to, carriers and excipients, such as permeation enhancers which promote transdermal absorption of the active ingredient after transdermal administration.

The amount of active ingredient absorbed depends on many factors. These factors include the active ingredient concentration, the active ingredient delivery vehicle, the skin contact time, the area of the skin dosed, the ratio of the ionized and unionized forms of the active ingredient at the pH of the absorption site, the molecular size of the active ingredient molecule, and the active ingredient's relative lipid solubility.

### Transdermal Devices

The transdermal device for delivering the active ingredients described herein can be of any type known in the art, including the monolithic, matrix, membrane, and other types typically useful for administering active ingredients by the transdermal route. Such devices are disclosed in U.S. Pat. Nos. 3,996,934; 3,797,494; 3,742,951; 3,598,122; 3,598,123; 3,731,683; 3,734,097; 4,336,243; 4,379,454; 4,460,372; 4,486,193; 4,666,441; 4,615,699; 4,681,584; and 4,558,580 among others; the disclosures of which are incorporated herein by reference.

These devices tend to be flexible, adhere well to the skin, and have a polymeric backing (covering) that is impermeable to the active ingredient to be delivered, so that the active ingredient is administered uni-directionally through the skin. The active ingredient, or pharmaceutically acceptable salt thereof, is typically present in a solution or dispersion, which can be in the form of a gel, a solution, or a semi-solid, and which aids in active ingredient delivery through the stratum corneum of the epidermis and to the dermis for absorption.

### Membrane Devices

Membrane devices typically have four layers: (1) an impermeable backing, (2) a reservoir layer, (3) a membrane layer (which can be a dense polymer membrane or a microporous membrane), and (4) a contact adhesive layer which either covers the entire device surface in a continuous or discontinuous coating or surrounds the membrane layer. Examples of materials that may be used to act as an impermeable layer are high, medium, and low density polyethylene, polypropylene, polyvinylchloride, polyvinylidene chloride, polycarbonate, polyethylene terepthalate, and polymers laminated or coated with aluminum foil. Others are disclosed in the standard transdermal device patents mentioned herein. In certain embodiments in which the reservoir layer is fluid or is a polymer, the outer edge of the backing layer can overlay the edge of the reservoir layer and be sealed by adhesion or fusion to the diffusion membrane layer. In such instances, the reservoir layer need not have exposed surfaces.

The reservoir layer is underneath the impermeable backing and contains a carrier liquid, typically water and/or an alcohol, or polyol or ester thereof, and may or may not contain the active ingredients. The reservoir layer can include diluents, stabilizers, vehicles, gelling agents, and the like in addition to the carrier liquid and active ingredients.

The diffusion membrane layer of the laminate device can be made of a dense or microporous polymer film that has the requisite permeability to the active ingredient and the carrier liquid. Preferably, the membrane is impermeable to ingredients other than the active ingredient and the carrier liquid, although when buffering at the skin surface is desired, the membrane should be permeable to the buffer in the composition as well. Examples of polymer film that may be used to make the membrane layer are disclosed in U.S. Pat. Nos. 3,797,454 and 4,031,894, each herein incorporated by reference. The preferred materials are polyurethane, ethylene vinyl alcohol polymers, and ethylene/vinyl acetate.

One embodiment of the invention relates to a transdermal composition comprising:

(1) an impermeable backing,
(2) a reservoir layer comprising the active ingredient in a buffer solution optional further comprising a penetration enhancer.
(3) a membrane layer (which can be a dense polymer membrane or a microporous membrane), and
(4) a contact adhesive layer which either covers the entire device surface in a continuous or discontinuous coating or surrounds the membrane layer.

In another embodiment the composition above comprised in the reservoir the active ingredient in phosphate buffered saline solution.

In a further embodiment the hydroxybenzoate salt of the active ingredient is used in the composition above.

In yet another embodiment the active ingredient in the reservoir of the composition above is present in a concentration of from 30 to 200 mg per gram of saline solution. In yet a further embodiment the concentration of the active ingredient in the reservoir of the composition above is 35 or 135 mg per gram of saline solution.

### Monolithic Matrices

The second class of transdermal systems is represented by monolithic matrices. Examples of such monolithic devices are U.S. Pat. No. 4,291,014, U.S. Pat. No. 4,297,995, U.S. Pat. No. 4,390,520, and U.S. Pat. No. 4,340,043, each herein incorporated by reference. Others are known to those of ordinary skill in this art.

Monolithic and matrix type barrier transdermal devices typically include:

(1) Porous polymers or open-cell foam polymers, such as polyvinyl chloride (PVC), polyurethanes, polypropylenes, and the like;

(2) Highly swollen or plasticized polymers such as cellulose, HEMA or MEMA or their copolymers, hydroxypropyl methylcellulose (HPMC), hydroxyethyl methylcellulose (HEMC), and the like, polyvinyl alcohol (PVA)/polyvinylpyrollidone (PVP), or other hydrogels, or PVC, polyurethane, ethylene/vinyl acetate, or their copolymers;

(3) Gels of liquids, typically including water and/or hydroxyl-containing solvents such as ethanol, and often containing gelling agents such PVP, carboxymethylcellulose (CMC), hydroxypropylcellulose such as sold under the tradename Klucel®, HPMC, alginates, kaolinate, bentonite, or montmorillonite, other clay fillers, stearates, silicon dioxide particles, and the like;

(4) Nonwoven materials made of textiles, celluloses, polyurethanes, polyester, or other fiber;

(5) Sponges, which can be formed from natural or foamed polymers; and

(6) Adhesives, ideally dermatologically-acceptable pressure sensitive adhesives, for example, silicone adhesives or acrylic adhesives.

The various components for the transdermal compositions are described in more detail below.

### Polymeric Barrier Materials

Representative polymeric barrier materials include, but are not limited to:

Polycarbonates, such as those formed by phosgenation of a dihydroxy aromatic such as bisphenol A, including materials are sold under the trade designation Lexan® (the General Electric Company);

Polyvinylchlorides, such as Geon® 121 (B. G. Goodrich Chemical Company);

Polyamides (“nylons”), such as polyhexamethylene adipamide, including NOMEX® (E. I. DuPont de Nemours & Co.).

Modacrylic copolymers, such as DYNEL®, are formed of polyvinylchloride (60 percent) and acrylonitrile (40 percent), styrene-acrylic acid copolymers, and the like.

Polysulfones, for example, those containing diphenylene sulfone groups, for example, P-1700 (Union Carbide Corporation).

Halogenated polymers, for example, polyvinylidene fluoride, such as Kynar® (Pennsalt Chemical Corporation), polyvinylfluoride, such as Tedlar® (E. I. DuPont de Nemours & Co.), and polyfluorohalocarbons, such as Aclar® (Allied Chemical Corporation).

Polychlorethers, for example, Penton® (Hercules Incorporated), and other thermoplastic polyethers.

Acetal polymers, for example, polyformaldehydes, such as Delrin® (E. I. DuPont de Nemours & Co.).

Acrylic resins, for example, polyacrylonitrile, polymethyl methacrylate (PMMA), poly n-butyl methacrylate, and the like.

Other polymers such as polyurethanes, polyimides, polybenzimidazoles, polyvinyl acetate, aromatic and aliphatic, polyethers, cellulose esters, e.g., cellulose triacetate; cellulose; colledion (cellulose nitrate with 11% nitrogen); epoxy resins; olefins, e.g., polyethylene, polypropylene; polyvinylidene chloride; porous rubber; cross linked poly(ethylene oxide); cross-linked polyvinylpyrrolidone; cross-linked poly(vinyl alcohol); polyelectrolyte structures formed of two ionically associated polymers of the type as set forth in U.S. Pat. Nos. 3,549,016 and 3,546,141, herein incorporated by reference with regard to such polymer compositions; derivatives of polystyrene such as poly(sodium styrenesulfonate) and poly(vinylbenzyltrimethyl-ammonium chloride); poly(hydroxyethylmethacrylate); poly(isobutylvinyl ether), and the like, may also be used. A large number of copolymers which can be formed by reacting various proportions of monomers from the above list of polymers are also useful.

If the membrane or other barrier does not have a sufficiently high flux, the thickness of the membrane or barrier can be reduced. However, the thickness should not be reduced to the point where it is likely to tear, or to a point where the amount of active ingredient which can be administered is too low.

### Adhesives

The transdermal drug delivery compositions typically include a contact adhesive layer to adhere the device to the skin. The active agent may, in some embodiments, reside in the adhesive.

Exemplary adhesives include polyurethanes; acrylic or methacrylic resins such as polymers of esters of acrylic or methacrylic acid with alcohols such as n-butanol, n-pentanol, isopentanol, 2-methylbutanol, 1-methylbutanol, 1-methylpentanol, 2-methylpentanol, 3-methylpentanol, 2-ethylbutanol, isooctanol, n-decanol, or n-dodecanol, alone or copolymerized with ethylenically unsaturated monomers such as acrylic acid, methacrylic acid, acrylamide, methacrylamide, N-alkoxymethyl acrylamides, N-alkoxymethyl methacrylamides, N-tertbutylacrylamide, itaconic acid, vinylacetate, N-branched alkyl maleamic acids wherein the alkyl group has 10 to 24 carbon atoms, glycol diacrylates, or mixtures of these; natural or synthetic rubbers such as styrenebutadiene, butylether, neoprene, polyisobutylene, polybutadiene, and polyisoprene; polyvinylacetate; unreaformaldehyde resins; phenolformaldehyde resins; resorcinol formaldehyde resins, cellulose derivatives such as ethylcellulose, methylcellulose, nitrocellulose, cellulose acetatebutyrate, and carboxymethyl cellulose; and natural gums such as guar, acacia, pectins, starch, dextrin, albumin, gelatin, casein, etc. The adhesives can be compounded with tackifiers and stabilizers, as is well known in the art.

Representative silicone adhesives include silicone elastomers based on monomers of silanes, halosilanes, or C₁₋₁₈ alkoxysilanes, especially polydimethylsiloxanes which may be used alone or formulated with a silicone tackifier or silicone plasticizer which are selected from medically acceptable silicone fluids, i.e. non-elastomeric silicones based on silanes, halosilanes or C₁₋₁₈ alkoxysilanes. Typical silicone adhesives are available from Dow Corning under the tradename SILASTIC®.

### Liquid Vehicles

Transdermal compositions can include a variety of components, including a liquid vehicle, typically a C₂₋₄ alkanol such as ethanol, isopropanol, n-propanol, butanol, a polyalcohol or glycol such as propylene glycol, butylene glycol, hexylene glycol, ethylene glycol, and/or purified water. The vehicle is typically present in an amount of between about 5 and about 75% w/w, more typically, between about 15.0% and about 65.0% w/w, and, preferably, between about 20.0 and 55.0% w/w.

Water augments the solubility of hydrophilic active agents in the composition, and accelerates the release of lipophilic active agents from a composition. Alcohols, such as ethanol, increase the stratum corneum liquid fluidity or function to extract lipids from the stratum corneum. As discussed herein, the glycols can also act as permeation enhancers.

### Permeation Enhancers

Successful transdermal delivery depends among others on sufficient flux of the active ingredient across skin, and sufficient surface area of skin, to produce an efficacious plasma concentration of the active ingredient. For reasons of consumer acceptance, the practical surface area of a transdermal system is limited from approximately 4 to 100 cm². With this limitation on surface area, the therapeutic transdermal administration of many active ingredients requires an increase in the inherent skin permeability to obtain the necessary flux. Accordingly, active ingredients have been developed which enhance percutaneous absorption of the active ingredients to be administered.

Permeation enhancers are described, for example, in U.S. Pat. Nos. 5,785,991; 4,764,381; 4,956,171; 4,863,970; 5,453,279; 4,883,660; 5,719,197, and in the literature “Pharmaceutical Skin Penetration Enhancement”, J. Hadgraft, Marcel Dekker, Inc. 1993; “Percutaneous Absorption”, R. Bronaugh, H. Maibach, Marcel Dekker, Inc. (1989), B. W. Barry, “Penetration Enhancers in Skin Permeation”, Proceedings of the 13th international Symposium on Controlled Release of Bioactive Materials, ed. by Chaudry & Thies, Controlled Release Society, Lincolnshire, Ill., pp. 136-137 (1986), and Cooper & Berner, “Penetration Enhancers”, in The Transdermal Delivery of Ingredients, Vol. II ed. by Kydonieus and Berner, CRC Press, Boca Raton, Fla. pp. 57-62 (1986), the contents of each of which are hereby incorporated by reference.

The permeation enhancers should both enhance the permeability of the stratum corneum, and be non-toxic, non-irritant and non-sensitizing on repeated exposure. Representative permeation enhancers include, for example, sucrose monococoate, glycerol monooleate, sucrose monolaurate, glycerol monolaurate, diethylene glycol monoalkyl ethers such as diethylene glycol monoethyl or monomethyl ether (Transcutol® P), ester components such as propylene glycol monolaurate, methyl laurate, and lauryl acetate, monoglycerides such as glycerol monolaurate, fatty alcohols such as lauryl alcohol, and 2-ethyl-1,3 hexanediol alone or in combination with oleic acid.

In one embodiment, the transdermal compositions are provided with skin permeation enhancing benefits by combining the active ingredients with saturated fatty alcohols, or forming salts of the active ingredients with one or more fatty acids, such as those of the formula CH₃—(CH₂)ₙ—CH₂OH or CH₃—(CH₂)ₙ—CH₂COOH respectively, in which n is an integer from 8 to 22, preferably 8 to 12, most preferably 10, or an unsaturated fatty alcohol or fatty acid given by the formula CH₃—(CₙH_{2(n-x)—OH or CH}₃—(CₙH₂₍ₙ₋ₓ₎—COOH respectively in which n is an integer from 8 to 22 and x is the number of double bonds; and preferably also a second component that is a monoalkyl ether of diethylene glycol, preferably diethylene glycol monoethyl ether or diethylene glycol monomethyl ether, in a vehicle or carrier composition, integrated by an C₁₋₄ alkanol, preferably ethanol; a polyalcohol, preferably propylene glycol and purified water.

A binary system including a combination of oleic acid or oleic alcohol and a lower alcohol, or a combination of a lower alkyl ester of a polycarboxylic acid, an aliphatic monohydroxy alcohol and an aliphatic diol, can be used.

Representative permeation enhancers include fatty alcohols and fatty acids, and monoalkyl ethers of diethylene glycol such as diethylene glycol monoethyl ether or diethylene glycol monomethyl ether. The fatty alcohols are typically present in an amount of between about 0.1 and about 20.0% w/w, preferably between about 0.2 and about 10.0% w/w, and more preferably, between about 0.4 and about 3.0% w/w. The diethylene glycol monoalkyl ethers are typically present in an amount up to 40.0% w/w, preferably between about 0.2 and 25.0% w/w, and, more preferably, between about 2.0 and about 8.0% w/w.

Although not wishing to be bound to a particular theory, it is believed that the mechanism by which certain permeation enhancers function to enhance permeability of the active agents through the stratum corneum is as follows:

The fatty alcohol is mainly distributed to the stratum corneum because of its lipophilicity and interacts with the stratum corneum lipids.

The diethylene glycol monoalkyl ethers dissolve both hydrophilic and a lipophilic active agents therein, and facilitates the penetration of the active agents to the skin.

Glycols, such as propylene glycol, act as a cosolvent of the active agents, and thus increase their solubility in the composition. Further, they tend to solvate the intracellular keratin of the stratum corneum, and thus enhance drug mobility and skin hydration.

### Gelling Agents

Gelling agents, such as carbomer, carboxyethylene or polyacrylic acid such as Carbopol® 980 or 940 NF, 981 or 941 NF, 1382 or 1342 NF, 5984 or 934 NF, ETD 2020, 2050, 934P NF, 971 P NF, 974P NF, Noveon® AA-1 USP, etc; cellulose derivatives such as ethylcellulose, hydroxypropylmethylcellulose (HPMC), ethylhydroxyethylcellulose (EHEC), carboxymethylcellulose (CMC), hydroxypropylcellulose (HPC) (Klucel®, different grades), hydroxyethylcellulose (HEC) (Natrosol® grades), HPMCP 55, Methocel® grades, etc; natural gums such as arabic, xanthan, guar gums, alginates, etc; polyvinylpyrrolidone derivatives such as Kollidon® grades; polyoxyethylene polyoxypropylene copolymers such as Lutrol® F grades 68, 127, etc; others like chitosan, polyvinyl alcohols, pectins, veegun grades, and the like, can also be present. Those of the skill in the art know of other gelling agents or viscosants suitable for use in the present invention.

Representative gelling agents include, but are not limited to, Carbopol® 980 NF, Lutrol® F 127, Lutrol® F 68 and Noveon® AA-1 USP. The gelling agent is present from about 0.2 to about 30.0% w/w, depending on the type of polymer.

### Preservatives

The transdermal compositions can also include one or more preservatives and/or antioxidants. Representative preservatives include quaternary ammonium salts such as lauralkonium chloride, benzalkonium chloride, benzododecinium chloride, cetyl pyridium chloride, cetrimide, domiphen bromide; alcohols such as benzyl alcohol, chlorobutanol, o-cresol, phenylethyl alcohol; organic acids or salts thereof such as benzoic acid, sodium benzoate, potassium sorbate, parabens; or complex forming agents such as ethylenediaminetetraacetic acid (EDTA). Representative antioxidants include butylhydroxytoluene, butylhydroxyanisole, ethylenediaminetetraacetic acid and its sodium salts, D,L-alpha tocoferol.

### Other Components

Other components may include diluents such as cellulose, microcrystalline cellulose, hydroxypropyl cellulose, starch, hydroxypropylmethyl cellulose and the like. Excipients can be added to adjust the tonicity of the composition, such as sodium chloride, glucose, dextrose, mannitol, sorbitol, lactose and the like. Acidic or basic buffers can also be added to control the pH. Co-solvents or solubilizers such as glycerol, polyethylene glycols, polyethylene glycols derivatives, polyethylene glycol 660 hydroxystearate (Solutol HS15 from BASF), butylene glycol, hexylene glycol, and the like, can also be added.

### Controlled Release of the Active Agent

The administration of the active agent can be controlled by using controlled release compositions, which can provide rapid or sustained release, or both, depending on the compositions.

There are numerous particulate drug delivery vehicles known to those of skill in the art which can include the active ingredients, and deliver them in a controlled manner. Examples include particulate polymeric drug delivery vehicles, for example, biodegradable polymers, and particles formed of non-polymeric components. These particulate drug delivery vehicles can be in the form of powders, microparticles, nanoparticles, microcapsules, liposomes, and the like. Typically, if the active agent is in particulate form without added components, its release rate depends on the release of the active agent itself. In contrast, if the active agent is in particulate form as a blend of the active agent and a polymer, the release of the active agent is controlled, at least in part, by the removal of the polymer, typically by dissolution or biodegradation.

In one embodiment, the compositions can provide an initial rapid release of the active ingredient followed by a sustained release of the active ingredient. U.S. Pat. No. 5,629,011 provides examples of this type of composition and is incorporated herein by reference in its entirety. There are numerous transdermal compositions that use transdermal delivery to deliver nicotine in a time-release manner (such as rate-controlling membranes), including currently marketed nicotine replacement therapies. These are also suitable for administering the metanicotines described herein.

### Semi-Solid Dosage Forms

In one embodiment, the transdermal dosage form is not a “patch,” but rather, a semisolid dosage form such as a gel, cream, ointment, liquid, etc. In this embodiment, one can augment patient's compliance and cover a broader surface area than can be covered with a patch.

In this embodiment, particularly when used for pain treatment, the dosage form can include other active and inactive components typically seen in semisolid dosage forms used to treat pain. These include, but are not limited to, menthol, wintergreen, capsaicin, aspirin, NSAIDs, narcotic agents (e.g. fentanyl), alcohols, oils such as emu oil, and solvents such as DMSO.

### IV. Iontophoresis

In addition to delivery via transdermal drug delivery devices and semi-solid dosage forms, the active ingredients can also be delivered via iontophoresis. Iontophoresis is a non-invasive method of propelling high concentrations of a charged substance, such as the active ingredients described herein, transdermally by repulsive electromotive force. The technique involves using a small electrical charge applied to an iontophoretic chamber containing a similarly charged active agent and its vehicle. The skin's permeability is altered upon application of the charge, and this increases migration of the active ingredient into the epidermis.

In use, one or two chambers are filled with a solution containing one or more of the active agents and a solvent (the “vehicle”). The positively charged chamber (“the anode”) will repel a positively charged chemical into the skin. The negatively charged chamber (“the cathode”), will repel a negatively charged chemical into the skin. Because the active ingredients are cationic, they are administered iontophoretically via the anode.

Iontophoresis can be used to transdermally deliver the active agents, using active transportation within an electric field, typically by electromigration and electroosmosis. These movements are typically measured in units of chemical flux, commonly μmol/cm²*h.

The isoelectric point of the skin is approximately 4. Under physiological conditions, where the surface of the skin is buffered at or near 7.4, the membrane has a net negative charge, and electroosmotic flow is from anode (−) to cathode (+). Electroosmosis augments the anodic delivery of the (positively charged) active agents described herein.

There are a number of factors that influence iontophoretic transport, including skin pH, the concentration and characteristics of the active agent, ionic competition, molecular size, current, voltage, time applied and skin resistance. The current density of the treatment electrode is perhaps the most important variable, relative to the degree of ion transfer. Comparable iontophoretic doses delivered at low currents over longer periods are more effective than those delivered by high currents over short periods.

Iontophoresis devices include two electrodes, which are typically attached to a patient, each connected via a wire to a microprocessor controlled electrical instrument. The active agents are placed under one or both of the electrodes, and are delivered into the body as the instrument is activated.

The instrument is typically designed to regulate both current flow and application time. Examples of such instruments are described in U.S. Pat. Nos. 5,254,081, and 5,431,625, the contents of which are hereby incorporated by reference. Power for these devices is usually provided by DC batteries, which when providing power for the microprocessor controlled circuitry allow application of a voltage to the electrodes to create a regulated current flow.

Wearable iontophoretic systems have been developed in which the electrical circuitry and power supplied are integrated into a single patch. These systems are advantageous in that they do not have external wires, and they are much smaller in size. Examples of such systems can be found in U.S. Pat. Nos. 5,358,483; 5,458,569; 5,466,217; 5,605,536; and 5,651,768, the contents of which are hereby incorporated by reference.

Typically, ions are delivered into the body from an aqueous drug reservoir contained in the iontophoretic device, and counter ions of opposite charge are delivered from a “counter reservoir.” Solutions containing the active ingredient, and also solutions of the counter ions, can be stored remotely and introduced to an absorbent layer of the iontophoresis electrode at the time of use. Examples of such systems are described in U.S. Pat. Nos. 5,087,241; 5,087,242; 5,846,217; and 6,421,561, the contents of which are hereby incorporated by reference. Alternatively, as described in U.S. Pat. No. 5,685,837, herein incorporated by reference, the active agents can be pre-packaged in dry form into the electrode(s). This approach requires a moisture activation step at the time of use.

Solutions of the active agents can be co-packaged with the iontophoretic device, ideally positioned apart from the electrodes and other metallic components until the time of use. This technique, and suitable devices, are described, for example, in U.S. Pat. Nos. 5,158,537; 5,288,289; 5,310,404; 5,320,598; 5,385,543; 5,645,527; 5,730,716; and 6,223,075, each of which is incorporated by reference. In these devices, a co-packaged electrolyte constituent liquid is stored remotely from the electrodes, in a rupturable container and a mechanical action step at the time of use induces a fluid transfer to a receiving reservoir adjacent to the electrodes. These systems enable precise fluid volumes to be incorporated at the time of manufacture to avoid overfilling.

In addition to solutions, the active agents can be present in a pre-formed gel, as described in U.S. Pat. No. 4,383,529, incorporated by reference. Thus, a preformed gel containing the active agent can be transferred into an electrode receptacle at the time of use. This system can be advantageous in that it provides a precise pre-determined volume of the gel, thus preventing over-filling. Further, since the active agent is present in a gel composition, it is less likely to leak during storage or transfer.

### V. Methods of Treatment

The transdermal compositions can be used to treat or prevent a condition or disorder in a subject susceptible to such a condition or disorder. Treatment includes amelioration of the symptoms of a disorder. As used herein, the terms “prevention” or “prophylaxis” include inhibition of the progression of a disorder, including but not limited to providing protective effects, inhibiting or delaying the onset of a disorder, or amelioration of the reoccurrence of a disorder. The method involves administering an effective amount of (2S)-(4E)-N-methyl-5-(3-(5-isopropoxypyridin)yl)-4-penten-2-amine, or one of its pharmaceutically acceptable salts, including fatty acid salts, such as stearic acid salts.

The instant active ingredients are modulators of the α4β2 NNR subtype, characteristic of the CNS, and can be used for preventing and/or treating various conditions or disorders, including those of the CNS, in subjects which have or are susceptible to such conditions or disorders, by modulation of α4β2 NNRs. The active ingredients have the ability to selectively bind to the α4β2 NNRs and express nicotinic pharmacology (e.g., to act as partial agonists, agonists, antagonists and/or inverse agonists). For example, the active ingredients described herein, when administered in effective amounts to patients in need thereof, provide some degree of prevention of the progression of the CNS disorder (i.e., providing protective effects), amelioration of the symptoms of the CNS disorder, and/or amelioration of the reoccurrence of the CNS disorder.

The active ingredient can be used to treat and/or prevent those types of conditions and disorders for which other types of nicotinic compounds have been proposed as therapeutics. See, for example, the references previously listed in the “Background of the Invention” section, as well as Williams et al., Drug News Perspec. 7(4): 205 (1994), Arneric et al., CNS Drug Rev. 1(1): 1-26 (1995), Arneric et al., Exp. Opin. Invest. Drugs 5(1): 79-100 (1996), Bencherif et al., J. Pharmacol. Exp. Ther. 279: 1413 (1996), Lippiello et al., J. Pharmacol. Exp. Ther. 279: 1422 (1996), Damaj et al., J. Pharmacol. Exp. Ther. 291: 390 (1999); Chiari et al., Anesthesiology 91: 1447 (1999), Lavand'homme and Eisenbach, Anesthesiology 91: 1455 (1999), Holladay et al., J. Med. Chem. 40(28): 4169-94 (1997), Bannon et al., Science 279: 77 (1998), PCT WO 94/08992, PCT WO 96/31475, PCT WO 96/40682, and U.S. Pat. Nos. 5,583,140 to Bencherif et al., 5,597,919 to Dull et al., 5,604,231 to Smith et al. and 5,852,041 to Cosford et al., the disclosures of which are incorporated herein by reference in their entirety.

The active ingredients and their pharmaceutical compositions are useful in the treatment and/or prevention of a variety of CNS disorders, including neurodegenerative disorders, neuropsychiatric disorders, neurologic disorders, and addictions. Among the disorders, diseases and conditions that the active ingredients and pharmaceutical compositions described herein can be used to treat and/or prevent are: age-associated memory impairment, mild cognitive impairment, pre-senile dementia, early onset Alzheimer's disease, senile dementia, dementia of the Alzheimer's type, Lewy body dementia, HIV-dementia, vascular dementia, Alzheimer's disease, stroke, AIDS dementia complex, attention deficit disorder, attention deficit hyperactivity disorder, dyslexia, schizophrenia, schizophreniform disorder, schizoaffective disorder, Parkinsonism including Parkinson's disease, Pick's disease, Huntington's chorea, tardive dyskinesia, hyperkinesia, progressive supranuclear palsy, Creutzfeld-Jakob disease, multiple sclerosis, amyotrophic lateral sclerosis, epilepsy, mania, anxiety, depression, panic disorders, bipolar disorders, generalized anxiety disorder, obsessive compulsive disorder, rage outbursts, Tourette's syndrome, autism, illicit and prescription drug and alcohol addiction, including tobacco addiction, obesity, cachexia, psoriasis, lupus, acute cholangitis, aphthous stomatitis, asthma, viral pneumonitis and arthritis (e.g., rheumatoid arthritis and osteoarthritis), endotoxaemia, sepsis, atherosclerosis, idiopathic pulmonary fibrosis and neoplasias.

One aspect of the present invention further provides use of the transdermal composition of the present invention comprising the active ingredient in the manufacturing of a medicament for treating or preventing disorders selected from the group consisting of Alzheimer's Disease, mild to moderate dementia of the Alzheimer's type, attention deficit disorder, attention deficit hyperactivity disorder, mild cognitive impairment, age-associated memory impairment, schizophrenia, and cognitive dysfunction in schizophrenia. Another aspect of the present invention includes the use of a transdermal composition of the present invention for treating or preventing disorders selected from the group consisting of Alzheimer's Disease, mild to moderate dementia of the Alzheimer's type, attention deficit disorder, attention deficit hyperactivity disorder, mild cognitive impairment, age-associated memory impairment, schizophrenia, and cognitive dysfunction in schizophrenia.

Another aspect of the present invention includes a method for the treatment or prevention of disorders selected from the group consisting of Alzheimer's Disease, mild to moderate dementia of the Alzheimer's type, attention deficit disorder, attention deficit hyperactivity disorder, mild cognitive impairment, age-associated memory impairment, schizophrenia, and cognitive dysfunction in schizophrenia comprising administering a composition of the present invention.

The active ingredients and their pharmaceutical compositions can be used to treat and/or prevent attentional disorders; to provide neuroprotection; to treat convulsions and multiple cerebral infarcts; to treat cognitive disorders, mood disorders, compulsions and addictive behaviors; to provide analgesia; to control inflammation (such as mediated by cytokines and nuclear factor kappa B) and treat inflammatory disorders; to provide pain relief (e.g., relief from acute pain, chronic pain, neurologic pain, neuropathic pain, female specific pain, post-surgical pain, or cancer pain); and to treat infections (as anti-infectious agents for treating bacterial, fungal and viral infections).

It is advantageous that the treatment or prevention of diseases, disorders and conditions occurs without appreciable adverse side effects (e.g., significant increases in blood pressure and heart rate, significant negative effects upon the gastro-intestinal tract, and significant effects upon skeletal muscle). The active ingredients and compositions described herein, when employed in effective amounts, can modulate the activity of the α4β2 NNRs without appreciable interaction with the NNR subtypes that characterize the human ganglia (as demonstrated by their lack of ability to elicit nicotinic function in adrenal chromaffin tissue) or skeletal muscle (as demonstrated by their lack of ability to elicit nicotinic function in cell preparations expressing muscle-type NNRs). Thus, these active ingredients are capable of treating and/or preventing diseases, disorders and conditions without eliciting significant side effects associated with activity at ganglionic and neuromuscular sites. Thus, administration of the active ingredients provides a therapeutic window in which treatment of certain diseases, disorders and conditions is provided, and certain side effects are avoided. That is, an effective dose of the active ingredient is sufficient to provide the desired effects upon the disease, disorder or condition, but is insufficient (i.e., is not at a high enough level) to provide undesirable side effects.

### Smoking Cessation

The transdermal compositions described herein can also be used to replace traditional transdermal nicotine patch devices, or “patches”, used in connection with smoking cessation. Due to substantial interpatient variability of nicotine blood levels, it is unlikely that a single dose of nicotine replacement therapy can be optimal for all humans, and, accordingly, they can be provided in various dosage levels to accommodate different patients.

It is believed that the compositions described herein will provide an acceptable risk/benefit in terms of adequate safety and provision of smoking cessation therapy. In view of the interpatient variability inherent in smoking, it is believed that the patient-controlled, flexible treatment described herein will eliminate entirely, or at least substantially reduce, use of tobacco in humans who undergo such treatment. Forms of such tobacco use are well known and include cigarettes, cigars and pipes, as well as “smokeless” tobacco products such as tobacco to be chewed or otherwise placed and kept in the mouth of humans.

In this application, the active ingredients are typically present in an amount, per patch, of between about 1 and 200 milligrams. Patches are configured in various sizes to give a range of skin contact surface areas to modulate the dose absorbed. The appropriate dosage for each patient will be governed by the human's personal tolerance to nicotine, and the ability of the active ingredient to effectively cross the stratum corneum of the epidermis and be absorbed in sufficient quantity to elicit the desired therapeutic response. Thus, based on factors such as the human's body weight, personal tolerance, etc., a broad range of daily dose exposure is likely to be observed in the treated population.

In some embodiments, the patients may increase or decrease the amount of use, as required. Desirably, the concentration of the active ingredients is decreased over time, as the patient lowers his/her perceived need to smoke.

Long-term treatment may be appropriate for certain individuals who require longer periods of treatment. For example, it may require as long as, for example, up to one year after commencement of treatment, or as little as six to ten weeks (or less).

Because the therapy can be patient-controlled, it may be the patient who determines what levels of the active agents are required over the course of treatment, or at various stages of treatment, in order to abate, avoid, or minimize his/her need to smoke. For example, should the patient have a strong urge to use tobacco products, this might indicate to the patient that a higher dosage level is required. Patients may refer to and/or rely on a behavioral support program which may include, for example, printed instructions and/or labels, or other oral instructions, advice or information given by qualified individuals who have been trained in giving advice on methods of treatment using the compositions described herein. Additionally, the patient may consult with a health care provider. However, the patients themselves, once having the educational benefit and guidance of the behavioral support program and/or the health care provider, will ideally be able to assess their own progress and respond appropriately in adjusting their dosage levels.

Ideally, since the active ingredients are more selective than nicotine, this form of therapy will avoid the symptoms associated with other nicotine therapy, including, without limitation, irritability, restlessness, insomnia, drowsiness, difficulty concentrating (e.g. impaired task performance), anxiety, hunger, cravings, vomiting, diarrhea, cold sweat, blurred vision, difficulty hearing, mental confusion, weakness, and/or fainting.

In addition to smoking cessation, the compositions can also be used to treat other addictions, such as alcohol, narcotics (including opiates such as oxycodone, morphine, heroine, and the like), prescription drugs, gambling, and the like.

The following examples are provided to illustrate the present invention, and should not be construed as limiting thereof. In these examples, all parts and percentages are by weight, unless otherwise noted.

### EXAMPLES

### Example 1

### Measurement of Drug Absorption

In vitro drug permeation experiments through abdominal guinea pig skin can be performed to evaluate the ability of a transdermal composition to deliver an active ingredient in pharmaceutically useful levels to a patient in need of treatment. These can be performed using a diffusion chamber, such as a Franz Vertical Diffusion Cell. The skin can be obtained from female Guinea pigs, 8 to 16 months of age, ideally shaved and free of lesions. A section of full thickness abdominal skin can be surgically excised and mounted between the sections of a vertical diffusion cell with the epidermal layer facing up. A transdermal device can be applied over the epidermal layer while the dermal layer is in contact with a receptor solution of PBS (or other suitable isotonic solution), with or without a suitable quantity of polyoxyethylene 20 oleyl ether (Oleth 20) or other surfactant to prevent foaming, at pH 7.4). The receptor chamber can be maintained at a suitable temperature, for example, around 35° C., and the permeation studies conducted under occlusive or non-occlusive conditions. At given time points, samples can be withdrawn from the receptor solution and the receptor chamber refilled with fresh solution. The samples can be analyzed, for example, using a high performance liquid chromatography (HPLC) method.

Flux Determination

Transdermal flux (mcg/cm²/h) can be determined from the steady-state slope of the plot of the cumulative amount of the active ingredient(s) permeating through the skin versus time. After steady-state is established, the linear portion of the plot can be used to calculate the flux from the slope.

From the in vitro data, one can extrapolate reasonably the results in vivo, particularly if the same transdermal composition, but for the identity of the active, has been similarly compared in an in vitro and in vivo correlative manner.

### Example 2

### In Vitro Skin Penetration Study Using Pig-Ear

Bronaugh diffusion cells were used for in vitro penetration studies, which were carried out to determine the flux (i.e. the speed by which the compound penetrates skin) and total cumulated amount penetrated of ¹⁴C-active ingredient. The compound was dissolved in water at two concentrations, 35 and 135 mg/g. Pig-ear full-thickness skin (dermatomed to approx. 900 μm thick) was used as the membrane. The receiver compartment contained phosphate buffered saline (pH 7.4) and the temperature was maintained at 32° C. The compound solutions were applied onto the stratum corneum. Receiver compartment samples (2, 4, 8 12 and 14 h) were assayed by liquid scintillation counting. The transdermal flux (μg/cm²/h) can be determined from the steady-state slope of the plot of the cumulative amount of active ingredient permeating through the skin versus time. After steady-state was established, the linear portion of the plot was used to calculate the flux from the slope.

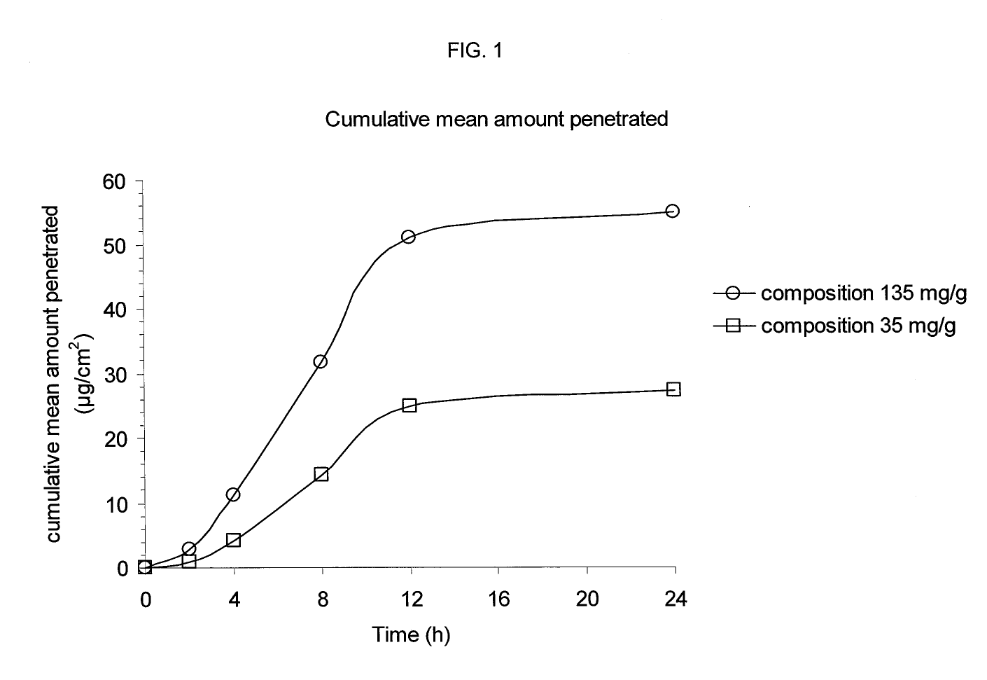

The cumulative penetrated amounts and derived steady state fluxes for both tested concentrations during a 24-hour period are presented in FIG. 1 and TABLE 1.

The active ingredient has advantageous properties for transdermal use as the steady state flux falls in the same range as for other active ingredients classified as successful transdermals, i.e. 0.2-40 μg/cm²/h (i.e. clonidine, estradiol, fentanyl, nicotine, scopolamine, testosterone). Higher flux can be expected at higher concentrations of active ingredient, e.g. 200 mg/g which is the maximal solubility in water.

TABLE 1

| | | Cumulative penetrated |
|---|---|---|
| Test concentration | Steady state flux | amount |
| mg/g water | (μg/cm²/h, average ± SD) | μg/cm² |
| 35 | 2.6 ± 3.6 | 27 ± 36 |
| 135 | 5.1 ± 9.0 | 55 ± 88 |

The specific pharmacological responses observed may vary according to and depending on the particular composition selected or whether there are present pharmaceutical carriers, as well as the type of composition and mode of administration employed, and such expected variations or differences in the results are contemplated in accordance with practice of the present invention.

Although specific embodiments of the present invention are herein illustrated and described in detail, the invention is not limited thereto. The above detailed descriptions are provided as exemplary of the present invention and should not be construed as constituting any limitation of the invention. Some modifications will be obvious to those skilled in the art, and all modifications that do not depart from the spirit of the invention are intended to be included with the scope of the appended claims.

1. A transdermal composition comprising:
(2S)-(4E)-N-methyl-5-(3-(5-isopropoxypyridin)yl)-4-penten-2-amine, or a pharmaceutically acceptable salt thereof, and
a pharmaceutically acceptable carrier.

2. The composition of claim 1, further comprising a permeation enhancer.

3. The composition of claim 1, further comprising one or more excipient, adhesive, diluent, binder, lubricant, glidant, disintegrant, carrier, surfactant, or mixture thereof.

4. The composition of claim 1, further comprising a rate-controlling membrane.

5. The composition of claim 1, wherein the composition is in a form of a transdermal patch.

6. A transdermal composition comprising:
(1) an impermeable backing,
(2) a reservoir layer comprising (2S)-(4E)-N-methyl-5-(3-(5-isopropoxypyridin)yl)-4-penten-2-amine or a pharmaceutically acceptable salt thereof in a buffer solution,
(3) a membrane layer, and
(4) a contact adhesive layer which either covers the entire device surface in a continuous or discontinuous coating or surrounds the membrane layer.

7. The composition of claim 6 further comprising a penetration enhancer.

8. The composition of claim 6 wherein the membrane layer is a dense polymer membrane or a microporous membrane.

9. The composition of claim 6, wherein the reservoir is a phosphate buffered saline solution.

10. The composition of claim 6, comprising (2S)-(4E)-N-methyl-5-(3-(5-isopropoxypyridin)yl)-4-penten-2-amine hydroxybenzoate.

11. The composition of claim 6, wherein the (2S)-(4E)-N-methyl-5-(3-(5-isopropoxypyridin)yl)-4-penten-2-amine or pharmaceutically salt thereof is in a concentration of from 30 to 200 mg per gram of saline solution.

12. A method for treating or preventing a central nervous system disorder, comprising administering the composition of claim 1.

13. The method of claim 12, wherein the central nervous system disorder is associated with an alteration in normal neurotransmitter release.

14. The method of claim 12, wherein the central nervous system disorder is selected from the group consisting of dyslexia, Parkinsonism including Parkinson's disease, Pick's disease, Huntington's chorea, tardive dyskinesia, hyperkinesia, progressive supranuclear palsy, Creutzfeld-Jakob disease, multiple sclerosis, amyotrophic lateral sclerosis, epilepsy, mania, anxiety, depression, panic disorders, bipolar disorders, generalized anxiety disorder, obsessive compulsive disorder, rage outbursts, Tourette's syndrome and autism.

15. A method for treating or preventing addiction, comprising administering the composition of claim 1.

16. The method of claim 15, wherein the addiction is smoking, alcoholism, gambling, or addiction to narcotics.

17. A method for treating, preventing, or alleviating pain, comprising administering the composition of claim 1.

18. The method of claim 17, wherein the type of pain is selected from the group consisting of acute pain, chronic pain, neurologic pain, neuropathic pain, female specific pain, post-surgical pain, inflammatory pain and cancer pain.

19. (canceled)

20. A method for transdermally administering (2S)-(4E)-N-methyl-5-(3-(5-isopropoxypyridin)yl)-4-penten-2-amine, or a pharmaceutically acceptable salt thereof, comprising administering the (2S)-(4E)-N-methyl-5-(3-(5-isopropoxypyridin)yl)-4-penten-2-amine, or a pharmaceutically acceptable salt thereof using iontophoresis.

21. The method of claim 20, wherein the (2S)-(4E)-N-methyl-5-(3-(5-isopropoxypyridin)yl)-4-penten-2-amine, or a pharmaceutically acceptable salt thereof is administered to treat central nervous system disorders.

22. The method of claim 21, wherein the central nervous system disorder is associated with an alteration in normal neurotransmitter release.

23. The method of claim 20, wherein the central nervous system disorder is selected from the group consisting of age-associated memory impairment, mild cognitive impairment, pre-senile dementia, early onset Alzheimer's disease, senile dementia, dementia of the Alzheimer's type, Lewy body dementia, HIV-dementia, vascular dementia, Alzheimer's disease, AIDS dementia complex, attention deficit disorder, attention deficit hyperactivity disorder, schizophrenia, schizophreniform disorder and schizoaffective disorder.

24. The method of claim 20, wherein the (2S)-(4E)-N-methyl-5-(3-(5-isopropoxypyridin)yl)-4-penten-2-amine, or a pharmaceutically acceptable salt thereof is administered to treat addiction.

25. The method of claim 24, wherein the addiction is smoking, alcoholism, gambling, or addiction to narcotics.

26. The method of claim 20, wherein the (2S)-(4E)-N-methyl-5-(3-(5-isopropoxypyridin)yl)-4-penten-2-amine, or a pharmaceutically acceptable salt thereof is administered to alleviate pain.

27. The method of claim 26, wherein the type of pain is selected from the group consisting of acute pain, chronic pain, neurologic pain, neuropathic pain, female specific pain, post-surgical pain, inflammatory pain and cancer pain.

28. A method for transdermal delivery of (2S)-(4E)-N-methyl-5-(3-(5-isopropoxypyridin)yl)-4-penten-2-amine, or a pharmaceutically acceptable salt thereof, at a steady state flux of between 1 to 50 ug/cm²/h.
